(19) United States
(12) Patent Application Publication
(10) Pub. No.: US 2023/0321343 A1
(43) Pub. Date: Oct. 12, 2023

(54) INFUSION OR TRANSFUSION SET AND SYSTEM COMPRISING AN INFUSION OR TRANSFUSION SET

(71) Applicant: B. Braun Melsungen AG, Melsungen (DE)

(72) Inventor: Andreas Katerkamp, Melsungen (DE)

(21) Appl. No.: 18/022,298

(22) PCT Filed: Aug. 27, 2021
(86) PCT No.: PCT/EP2021/073788

§ 371 Date: Feb. 21, 2023

(30) Foreign Application Priority Data
Aug. 31, 2020 (DE) 10 2020 210 986.9

(51) Int. Cl. A61M 5/142; A61M 39/24

(52) U.S. Cl. CPC A61M 5/14228; A61M 39/24

(57) ABSTRACT

An infusion or transfusion set for administering a liquid from a container using a pump. The infusion or transfusion set includes a branch that provides a fluid connection between a first supply line, a second supply line and a discharge line. The first supply line includes a liquid-retaining filter membrane having a breakdown pressure. At least the second supply line has a check valve. The check valve is opened for fluid passage in the direction towards the branch if the pressure difference at the check valve is greater than a threshold value that is less than the breakdown pressure of the liquid-retaining filter membrane.

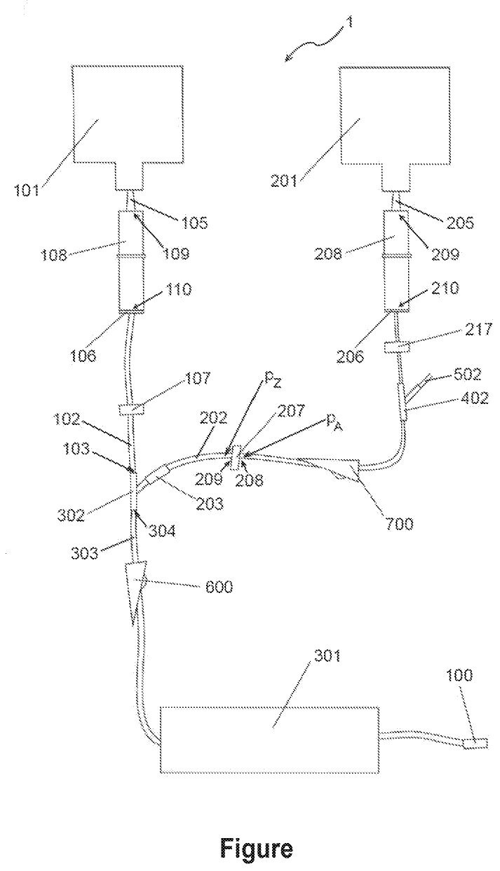

### CROSS-REFERENCE TO RELATED APPLICATIONS

This application is the United States national stage entry of International Application No. PCT/EP2021/073788, filed Aug. 27, 2021, and claims priority to German Application No. 10 2020 210 986.9, filed Aug. 31, 2020. The contents of International Application No. PCT/EP2021/073788 and German Application No. 10 2020 210 986.9 are incorporated by reference herein in their entireties.

### FIELD

Infusions and transfusions are performed for therapeutic purposes in human and veterinary medicine. Infusions and transfusions are used to administer liquids to a patient. For example, infusions may be used to administer liquid drugs (drug solutions, etc.).

### BACKGROUND

An infusion set or transfusion set is understood to be a product with which the administration of a medical infusion or the performance of a medical transfusion or the performance of a comparable administration of a liquid may be carried out. For example, the terms “infusion set”, “infusion or transfusion system”, “infusion system”, “infusion kit or transfusion kit” or “infusion kit” are also commonly used for an infusion set or transfusion set, wherein the use of the terms “infusion set”, “infusion system” and “infusion kit” are not intended to exclude the possibility that the product so designated may also be used, for example, to perform a transfusion.

An infusion set or transfusion set generally comprises a conduit configured as a tube and often a drip chamber. The infusion set or transfusion set may optionally include further components such as a flow controller for controlling the flow rate of the liquid, e.g. a roller clamp. The liquid to be administered in the course of an infusion or transfusion is provided in a container. The container may be, for example, an infusion bottle, an infusion bag, a blood bag, etc.

If a drip chamber is provided, it is typically connected to the container via a container connector so that the liquid may exit the container and enter the drip chamber. The inlet of the drip chamber is configured as a drop former, which causes the liquid to enter the drip chamber from the container in the form of droplets of normalized size. The container connector may, for example, be a piercing device such as a hollow mandrel that may be used to pierce a septum closing the container and that typically comprises a plurality of channels in its interior. Such a piercing device is commonly referred to as a “spike”. Other systems are also known for connecting the drip chamber to the container, such as coupling systems that do not allow the drip chamber and container to be separated once they have been connected. The drip chamber is in fluid communication with one end of the tube, so that liquid may enter the tube from the drip chamber. If a drip chamber is not used, the tube is connected directly to the container or a suitable container connector. The tube comprises a connection for the patient access port (e.g., venous cannula or venous catheter) at the other end. The connection for the patient access port is referred to below as the “patient connection”. The patient access port may optionally also be considered to be an element of the infusion set or transfusion set.

A drip chamber, as described, provides the connection between the tube and the container. Frequently, devices that ensure the ventilation of the system are integrated into the drip chamber. For this purpose, the drip chamber comprises, for example, a vent device with a manually operated or an automatic vent valve and a vent channel open to the interior of the drip chamber. In the prior art, different embodiments of the vent device based on different types of valves and with or without a vent filter are known, for example, manual vent devices that have a manually operated flap as a vent valve and automatic vent valves that comprise a check valve (non-return valve) as a vent valve. Alternatively, the manual or automatic vent device is not integrated into a drip chamber but is located at another suitable position of the fluid system. The present invention is compatible with manual and automatic vent devices. In particular, if the container in which the liquid to be administered is presented is collapsible, the use of a drip chamber or vent device may also be omitted because it is then not necessary to allow air to flow into the system for pressure equalization.

In addition to the gravity infusion or gravity transfusion technique, in which the liquid is delivered from the container to the patient access port solely by the action of gravity, the pump infusion or pump transfusion technique has become established. In pump infusion or pump transfusion, the liquid is conveyed using a pump. By using a pump, the administration of the liquid may be controlled in a better way. For example, the pump may be a peristaltic pump, which engages and periodically deforms a portion of the tube to create a peristaltic pumping motion. Such peristaltic pumps are advantageous because no components of the pump come into contact with the liquid such that there is no risk of contamination caused by the use of the pump. Furthermore, peristaltic pumps are easy to handle. In particular, it is easy to connect a tube to the pump and to disconnect this connection after the infusion is complete. For this purpose, peristaltic infusion or transfusion pumps frequently comprise a housing with an open channel or slot into which the tube is inserted.

In the course of administering an infusion or transfusion, it is necessary to prevent large amounts of air from entering the patient's body. Air entering the bloodstream, for example, may cause a life-threatening air embolism. To prevent air from entering the patient's body, for example, it is necessary to fill the tube with the liquid before beginning administration of the liquid to the patient. This preparatory step is often referred to as “priming” or “priming step”.

To prevent air from entering the patient's body, it is also necessary to ensure that the infusion or transfusion is terminated as soon as the liquid to be administered is used up. To facilitate the termination of the infusion or transfusion at the correct time, some of the infusion sets or transfusion sets on the market have a liquid-retaining filter membrane. This liquid-retaining filter membrane is located in the fluid channel through which the liquid passes from the container to the patient access port. For example, the liquid-retaining filter membrane may be located at the bottom of the drip chamber, i.e., near its exit. However, it may also be located elsewhere in the tube. When the liquid is used up to the point that there is essentially no liquid left in the portion of the fluid system between the liquid-retaining filter membrane and the container, a resistance to further flow of the liquid builds up at the liquid-retaining membrane. That is, the liquid-retaining membrane functions as a membrane that resists the flow of the column of liquid located in the tube below the liquid-retaining membrane and, in this sense, retains the liquid.

The liquid-retaining filter membrane has the function of preventing the passage of fluid if there is no liquid or only a low liquid level above the membrane. In this way, air is prevented from passing through the membrane such that it is ensured that air does not enter the patient's bloodstream.

This is achieved, for example, in that the membrane has a porous structure and the liquid passing through the membrane flows through the pores or through the channels formed by the pores. According to a theory, the function of such a membrane is explained as follows, although the present invention is not limited to ensuring that the liquid-retaining membrane functions according to this theory: According to the theory, if there is at least some amount of liquid above the membrane, or liquid is dropped onto the membrane from above, the weight of that liquid provides sufficient pressure to allow the liquid to flow through the pores or channels; or liquid is available to flow downstream into the pores or channels, such that the capillary effect described below does not come into play. If there is no liquid or only a slight excess of liquid above the membrane, the liquid is held in the pores or channels as a result of capillary forces and air may be prevented from flowing through the pores or channels. The situation may be imagined in such a way that the liquid in the pores or channels configures a meniscus and does not flow further. This capillary effect is also known as “capillary stop flow”. Liquid-retaining membranes that exploit this capillary effect and, optionally, those membranes that may have an analogous function by means of another effect are referred to as “air-stop membranes”. Preferably, the membrane comprises a hydrophilic material because the capillary effect is then more pronounced for the usually water-based liquid to be administered to the patient than in the case of a non-hydrophilic material.

The pressure difference between the top and bottom of the membrane, beyond which air can enter the membrane to displace the fluid held in the pores or channels by the capillary effect described above is called the “breakdown pressure”. The term “bubble point pressure” is also used for the breakdown pressure. In operation, in the case of a conventional infusion set, there is a column of fluid in the tube below the membrane, which creates a negative pressure on the membrane as a result of gravity, depending on the length of the fluid column. The breakdown pressure must exceed the negative pressure generated by the column of liquid to prevent the liquid column from flowing away and air from entering the tube. The dimensions of the membrane as well as its materials and structure are selected for gravity infusion and adapted to the infusion fluid and the length of the tubing so that the breakdown pressure is sufficient to allow the membrane to prevent the fluid column from flowing off. For example, the breakdown pressure for some commercially available infusion sets with a tube length of approximately 150 cm is typically at least 200 mbar (20 kPa). 200 mbar corresponds to a water column of 200 cm. Since the tube length and thus the liquid column are shorter, the flow is automatically stopped in the case of gravity infusion as soon as the container or drip chamber is empty. When using an infusion pump, the pump is automatically turned off for example as soon as the pressure difference between the top and the bottom of the membrane has a predetermined value that is lower than the breakdown pressure. For example, the pump may be set to shut off at a pressure drop of 17 kPa (corresponding to a pressure in the tube of −17 kPa compared to the pressure that would otherwise prevail during infusion).

In the course of a gravity infusion or gravity transfusion, the capillary effect described above may be exploited to stop the flow of fluid as soon as the container or drip chamber is empty. In the course of a pump infusion or pump transfusion, this effect may be exploited in a similar way by configuring the pump in such a way that an alarm signal is sent and/or the pumping of liquid is stopped as soon as the container or the drip chamber is empty. Usually, the pumps used have a pressure measuring device that can detect the pressure of the liquid in the pump inlet side portion of the tube. The pump inlet side portion (pump upstream portion) of the tube is understood to be the portion of the tube that is upstream of the pump. The liquid-retaining filter membrane is preferably located upstream of the pump. Therefore, in this case, the pump upstream portion of the tube is understood to be the portion between the pump and the liquid-retaining filter membrane. When the container or drip chamber is empty, pressure of the liquid in the pump upstream portion of the tube decreases. A control device that receives signals from the pressure measuring device is configured to send an acoustic and/or visual alarm signal and/or switch off the pump when it is determined that the infusion or transfusion has ended.

Frequently, multiple infusions or transfusions are administered to a patient in succession, for example because different active ingredient solutions are to be administered to the patient in succession by infusion or because the patient is to be administered an amount of fluid that exceeds the capacity of a typical container for infusion fluids or transfusion blood. Administering multiple infusions or transfusions in succession is also referred to as “sequential infusion” or “sequential transfusion”, respectively. To administer multiple infusions or transfusions sequentially to a patient, an initial infusion or transfusion is administered first. When the first infusion or transfusion is completed, another infusion is started by the medical staff. This procedure is repeated if more than two infusions are to be administered. In other words, when an infusion has been completed and, if necessary, an appropriate alarm signal has been sent from the pump and/or the pump has been switched off automatically, it is necessary for the medical staff to take further steps. Thus, performing sequential infusions is time-consuming and labour-intensive for the medical staff.

### SUMMARY

In view of the above-mentioned situation, one object of the invention is to provide an improved infusion set or transfusion set and an improved system comprising such an infusion sets or transfusion set.

The infusion set or transfusion set according to the invention is an infusion set or transfusion set for administering a liquid from a container using a pump. The infusion set or transfusion set comprises a branch, wherein fluid communication is provided between respective ends of a first supply line, a second supply line, and a discharge line via the branch. The first supply line is configured to be connected to the container at its end remote from the branch. At least the first supply line comprises a liquid-retaining filter membrane. At least the second supply line comprises a check valve. This check valve is closed for a passage of a fluid in the direction from the branch. Said check valve is configured to be closed also for a fluid passage in the direction towards the branch if the pressure difference Δp=p_{A}−p_{Z} between a pressure p_{A} present in the second supply line on the side of the check valve facing away from the branch and a pressure p_{Z} present in the second supply line on the side of the check valve facing towards the branch is less than a threshold value p_{R}, i.e. if Δp=p_{A}−p_{Z}<p_{R}. This check valve is further configured to be open for fluid passage in the direction towards the branch if the pressure difference Δp=p_{A}−p_{Z} between the pressure p_{A} present in the second supply line on the side of the check valve facing away from the branch and the pressure p_{Z} present in the second supply line on the side of the check valve facing towards the branch is greater than the threshold value p_{R}, i.e. if Δp=p_{A}−p_{Z}>p_{R}. p_{R} is thereby less than the breakdown pressure p_{Mem} of the liquid-retaining filter membrane. Such an infusion set or transfusion set may be used, for example, to automatically sequentially administer two liquids (e.g., infusion fluids) to a patient.

Preferably, the branch provides a fluid connection between an end of each of the first supply line, the second supply line, and the discharge line by forming the branch as a separate component, for example, as a Y-port, and connecting the first supply line, the second supply line, and the discharge line to the branch via one of their ends, respectively, such that a fluid connection between the first supply line, the second supply line, and the discharge line is provided via the branch. Alternatively, the second supply line may, for example, be connected to or formed integrally with a conduit at a junction such that this conduit forms the first supply line upstream of the second supply line (in the direction of flow when administering a liquid to a patient) as well as the discharge line downstream of the second supply line. The junction then constitutes the branch.

When this patent application refers to a check valve being closed to fluid passage in a particular direction, this means that the check valve shuts off the flow of a fluid in that direction and thus no fluid, or at least no significant amount of fluid, may flow through the check valve. When this patent application refers to a check valve being open for fluid passage in a particular direction, this means that the check valve does not shut off the flow of a fluid in that direction and thus fluid may flow through the check valve. In particular, the fluid may then flow freely through the check valve. Fluid is understood to mean gases, liquids and mixtures thereof.

According to the invention, the check valve has the function of allowing fluid to pass through the check valve and thus through the second supply line only under a certain condition (Δp>p_{R}) in one flow direction and preventing the passage of fluid otherwise. The passage of fluid is prevented if this condition is not met and, in particular, in the direction opposite to this flow direction. The flow direction is the direction from the check valve to the branch or, in other words, the direction to the patient connection. In other words, the function of the check valve may be described as follows: If fluid wants to flow through the check valve against the flow direction, i.e., if p_{Z} is greater than p_{A} and Δp is thus negative, the check valve will not allow the fluid to flow. If p_{A} is greater than p_{Z} to only such an extent that Δp is less than p_{R}, the check valve will also not allow the fluid to flow. If p_{A} is greater than p_{Z} to such an extent that Δp exceeds the predetermined determined threshold p_{R}, fluid may flow through the check valve and thus through the second supply line. The threshold value p_{R} thus corresponds to the valve opening pressure of the check valve.

Because of the arrangement and configuration of the check valve and the liquid-retaining filter membrane according to the invention, for example an automatic sequential administration of two liquids (e.g., infusion liquids) to a patient may be performed, wherein no separate action is required after completion of the administration of the first liquid and before commencement of the administration of the second liquid.

The second supply line and any other optional supply lines may also each include a liquid-retaining filter membrane. The liquid-retaining filter membrane of the first supply line has a breakdown pressure p_{Mem}. The breakdown pressures of the optional liquid-retaining filter membranes of the second, third, . . . n-th supply lines are designated as p_{Mem}⁽²⁾, p_{Mem}⁽³⁾, . . . , p_{Mem}⁽ⁿ⁾. By the term “n-th” is meant the ordinal number to the natural number n.

In preferred embodiments of the invention, the branch is a Y-port. A Y-port is a tubular element having three ports in fluid communication with each other. The three ports need not be symmetrically arranged.

In preferred embodiments of the invention, the first supply line is adapted to be connected to a first container at its end remote from the branch so that fluid may flow from the first container into the first supply line. Further, the second supply line is adapted to be connected at its end remote from the branch to a second container so that fluid may flow from the second container into the second supply line. In this way, by using the infusion set or transfusion set, a sequential infusion or transfusion may be carried out in a particularly advantageous manner, in which fluid is first administered to a patient as a first fluid from the first container and then a further fluid is administered as a second fluid from the second container using the pump. In this way, it is made possible to administer several fluids to a patient in succession without requiring any action on the part of the medical staff after completion of the administration of one fluid for the administration of the other fluid. This not only results in time and cost savings but also reduces the risk of error on the part of medical staff.

If the first or second supply line is designed as a tube, the respective end remote from the branch is the respective free tube end.

With an appropriately designed infusion set or transfusion set with more than two supply lines, the sequential administration of more than two fluids is possible in an analogous manner, which will be discussed in detail below.

In preferred embodiments of the invention, the threshold value p_{R} is predetermined to be greater than or equal to a predetermined minimum threshold value p_{Rmin} and/or less than or equal to a predetermined maximum threshold value p_{Rmax}, p_{Rmin} and p_{Rmax} are selected to allow safe use of the infusion and transfusion set in which undesired flow of fluid is reliably prevented. In particular, air should be prevented from passing through the liquid-retaining filter membrane. Typically, the breakdown pressure p_{Mem} of the liquid-retaining filter membrane is between 200 mbar and 3000 mbar. For such values of p_{Mem}, it has been found advantageous that p_{R} is at most 190 mbar, preferably at most 175 mbar, more preferably at most 150 mbar, and p_{Rmax} is thus 190 mbar, preferably 175 mbar, more preferably 150 mbar. If a liquid-retaining filter membrane with a small p_{Mem} value is used, p_{Rmax} is to be reduced accordingly, so that p_{Rmax} is preferably 10 mbar, more preferably 25 mbar, even more preferably 50 mbar less than p_{Mem}.

If the end of the second supply line is located higher than the end of the first supply line, there may be a corresponding contribution to the pressure conditions in the fluid system. Furthermore, pressure fluctuations, for example due to movement or deformation of the infusion set or transfusion set, may result in a contribution to the pressure conditions in the fluid system. If possible, it should be avoided that such contributions to the pressure conditions lead to fluid flowing unintentionally through the check valve of the second supply line, for example when fluid still flowing through the first supply line is administered to the patient. Particularly in view of these circumstances, it has proven advantageous that p_{R} is at least 25 mbar, preferably at least mbar, more preferably at least 50 mbar, and p_{Rmin} is thus 25 mbar, preferably 35 mbar, more preferably 50 mbar.

In preferred embodiments of the invention, the first supply line also has a check valve. This check valve is arranged between the branch and the liquid-retaining membrane. This check valve is closed for a passage of a fluid in the direction from the branch. In particular, this check valve offers the advantage that during and after priming of the infusion set or transfusion set, an unintentional backflow of the fluid in the direction towards the container may be prevented.

It is possible within the scope of the invention for the infusion set or transfusion set to comprise more than two supply lines, such that, in addition to the first supply line and the second supply line, a third supply line and, optionally, further supply lines are present.

If a third supply line is present, the second supply line has a liquid-retaining filter membrane associated with the second supply line. The third supply line has a check valve associated with the third supply line and may optionally have a liquid-retaining filter membrane associated with the third supply line. If a fourth supply line is additionally provided, the third supply line also comprises a liquid-retaining filter membrane associated with the third supply line. The fourth supply line has a check valve associated with the fourth supply line and may optionally have a liquid-retaining filter membrane associated with the fourth supply line. Generally, the first, second, . . . , (nₘₐₓ−1)-th supply lines each have a liquid-retaining filter membrane associated therewith, wherein there is a total of nₘₐₓ supply lines. Generally, the second, . . . , (nₘₐₓ)-th supply lines have a check valve associated with each of them, wherein there is a total of nₘₐₓ supply lines. nₘₐₓ is a natural number and denotes the number of supply lines.

The third and each additional supply line, referred to as the n-th supply line, may branch off from the branch, for example. However, it may also branch off at a point on the (n−1)-th supply line that is downstream of the liquid-retaining filter membrane of the (n−1)-th supply line, where n is an index that runs from 1 to nₘₐₓ and denotes the individual supply lines. For example, the third supply line may branch off from the branch. However, it may also branch off at a point on the second supply line that lies between the branch and the liquid-retaining filter membrane of the second supply line.

Δp⁽ⁿ⁾=p_{A}⁽ⁿ⁾−p_{Z}⁽ⁿ⁾ (n=2, 3, . . . , nₘₐₓ) denotes the pressure difference between a pressure p_{A}⁽ⁿ⁾ present in the n-th supply line on the side of the check valve associated with the n-th supply line facing away from the branch and a pressure p_{Z}⁽ⁿ⁾ present in the n-th supply line on the side of the check valve associated with the n-th supply line facing towards the branch. It is noted that, in particular, when describing embodiments in which there are only two supply lines, the subscript “2” in Δp⁽ⁿ⁾, p_{A}⁽ⁿ⁾, p_{Z}⁽ⁿ⁾ may be omitted for simplicity, i.e. Δp=Δp⁽²⁾, p_{A}=p_{A}⁽²⁾, p_{Z}=p_{Z}⁽²⁾.

Expressions such as “n=2, 3, . . . , nₘₐₓ” are used to indicate which values the index may adopt one after the other. For example, the expression “n=2, 3, . . . , nₘₐₓ” expresses that the index n runs through the values of all natural numbers from 2 (included) to nₘₐₓ. For example, if nₘₐₓ is 2, the expression means that n may only take the value 2. For example, if nₘₐₓ is 5, the expression means that n takes the values 2, 3, 4, and 5.

p_{Mem}⁽ⁿ⁾ (n=1, 2, . . . , nₘₐₓ−1) denotes the breakdown pressure of the liquid-retaining filter membrane associated with the n-th supply line. If the nₘₐₓ-th supply line also has a liquid-retaining filter membrane, its breakdown pressure may also be denoted as p_{Mem}^{(nmax)}.

The check valve (n=2, 3, . . . , nₘₐₓ) associated with the n-th supply line is always closed for a passage of a fluid in the direction from the branch. The check valve (n=2, 3, . . . , nₘₐₓ) associated with the n-th supply line is arranged to be closed for fluid passage in the direction towards the branch if the pressure difference Δp⁽ⁿ⁾ is less than a predetermined threshold value p_{R}⁽ⁿ⁾. The check valve (n=2, 3, . . . , nₘₐₓ) associated with the n-th supply line is further arranged to be open for fluid passage in the direction to the branch if the pressure difference Δp⁽ⁿ⁾ is greater than the threshold value p_{R}⁽ⁿ⁾.

The threshold values p_{R}⁽ⁿ⁾ (n=2, 3, . . . , nₘₐₓ) are predetermined such that p_{R}⁽ⁿ⁾ is greater than p_{R}⁽ⁿ⁻¹⁾, i.e., p_{R}⁽³⁾ is greater than p_{R}⁽²⁾, etc. The threshold values p_{R}⁽ⁿ⁾ (n=2, 3, . . . , nₘₐₓ) are further predetermined such that p_{R}⁽ⁿ⁾ is less than all p_{Mem}⁽ⁿ⁾ (n=1, 2, . . . , nₘₐₓ). Here, the inequality p_{Rmin}≤p_{R}⁽ⁿ⁾≤p_{Rmax} holds for n=nₘₐₓ and thus for all values that the index n adopts.

From the above description it is clear that the case nₘₐₓ≥3 is a homologous continuation of the case described further above of an infusion set or transfusion set with only a first supply line and a second supply line (nₘₐₓ=2), and in both cases the same concept according to the invention is realized.

The system according to the invention is a system comprising an infusion set or transfusion set according to the invention and a pump for delivering fluid through the supply lines and the discharge line.

In preferred embodiments, the pump is a peristaltic pump, in particular a peristaltic pump engaged with or adapted to be engaged with a portion of an outer wall of the discharge line. The peristaltic pump, when engaged with a portion of the outer wall of the discharge line, is capable of peristaltically deforming the discharge line and thereby pumping fluid through the infusion set or transfusion set to the patient access port.

In preferred embodiments, the system further comprises a control device and a pressure sensing device for sensing a measurement value corresponding to a pump upstream pressure of the pumped liquid. The pressure measuring device may, for example, be integrated into the pump or arranged at the pump inlet side of the pump in the same housing as the pump. The “pump inlet side” (“pump upstream side”) is the upstream side of the pump, i.e. the side from which liquid flows towards the pump. In operation, this is the side opposite the side in the direction towards the patient access port. The control device may be configured to send an alarm signal when the pressure on the pump upstream side falls below a threshold value p_{Alarm}. That is, the control device then sends a control signal to an alarm transmitter, such as a visual and/or audible signal transmitter. The signal transmitter may be integrated into the control device. Alternatively or additionally, the control device may be configured to stop pumping liquid when the pressure on the pump upstream side falls below the threshold value p_{Alarm}. That is, the control device then sends a control signal to the pump to stop pumping.

Preferably, the threshold value p_{Alarm} is selected such that no value Δp⁽ⁿ⁾ (n=2, 3, . . . , nₘₐₓ) is beyond a breakdown pressure p_{Mem}⁽ⁿ⁾ (n=1, 3, . . . , nₘₐₓ) when the pressure on the pump upstream side falls below the threshold value p_{Alarm}, so that an alarm is set off and/or the pumping of liquid is stopped before the pressure conditions prevailing in the fluid system cause a breakdown of a liquid-retaining membrane, i.e. the undesired passage of air through the liquid-retaining membrane.

### BRIEF DESCRIPTION OF THE DRAWING

Further features, expediencies, and advantages of the invention are described below with reference to exemplary embodiments with reference to the attached FIGURE.

The FIGURE shows an infusion set or transfusion set 1 according to a first embodiment of the invention as well as a pump 301 and two containers 101, 201.

### DETAILED DESCRIPTION

The infusion set or transfusion set 1 is for administering liquid initially from the container 101 using a pump 301, i.e., the pump 301 delivers the liquid through the infusion set or transfusion set 1 to the patient connection 100. The patient connection 100 provides a connection for a patient access port. The patient access port is not shown in the FIGURE. The patient access port may be, for example, a venous cannula, a venous catheter, etc. The patient access port may optionally also be considered part of the infusion set or transfusion set.

The infusion set or transfusion set 1 comprises a conduit system through which liquid may be delivered to the patient connection 100.

The conduit system preferably comprises tubes.

The conduit system comprises a branch 302, i.e. an element through which a plurality of conduits are interconnected such that a fluid connection exists between all conduits. A fluid connection is therefore provided via the branch 302 between the first supply line 102, the second supply line 202, optionally further supply lines, and the discharge line 303.

A first supply line 102, a second supply line 202 and a discharge line 303 are connected to the branch 302 via one of their ends 103, 203, 304, respectively. In alternative embodiments not shown in the FIGURE, more than three supply lines are connected to the branch 302, for example, a third supply line in addition to the first supply line 102 and the second supply line 202.

The terms “supply line” and “discharge line” express that when a liquid is administered to a patient, i.e., when the liquid flows through the infusion set or transfusion set to the patient connection, the liquid flows through a supply line from a container to the branch and through a discharge line from the branch to the patient connection.

In the embodiment shown in the FIGURE, the branch 302 is a Y-port, i.e., a Y-shaped tubular element, preferably made of plastic, to which the supply lines 102, 202 and the discharge line 303 are connected. The connection may, for example, be made by a push-fit, adhesive, or welded connection. In alternative embodiments with two supply lines and one discharge line, the branch is a T-shaped tubular element. In yet other embodiments, the branch is not formed by a separate element, but is formed, for example, in that the second supply line is branched directly from a line, i.e. the second supply line is connected to or formed integrally with the line at a junction. This line then forms the first supply line upstream of the second supply line (in the direction of flow when administering a liquid to a patient) and the discharge line downstream of the second supply line. The junction then constitutes the branch.

In embodiments in which more than three lines are connected to the branch, the branch is configured accordingly, for example as a cross-shaped tubular element or as a tubular element with two tubes extending from a tube at an acute angle in the case of three supply lines. Also in embodiments in which more than three supply lines are connected to the branch, it is possible that the branch is not formed by a separate element, but rather, for example, in the case of three supply lines, the second and third supply lines are connected to or formed integrally with one line such that this line forms the first supply line upstream of the second or third supply line and the discharge line downstream of the second or third supply line.

The first supply line 102 may be connected to a container 101 at its end 105 remote from the branch 302. The container 101 includes a liquid to be administered to the patient.

In the embodiment shown in the FIGURE, the first supply line 102 comprises a tube having two ends, one end 103 of which is connected to the branch 302 and the other end of which is connected to the outlet 110 of a drip chamber 108. The outlet 110 may allow liquid from the drip chamber 108 to flow into the tube. The drip chamber further comprises an inlet 109 configured as a drop former, which allows the liquid from the container 101 to enter the drip chamber 108 drop by drop. The drip chamber 108 comprises a container connector 105 in the region of its entrance. Through the container connector 105, the first supply line 102 may be connected to the container 101 such that the liquid may flow from the container 101 into the first supply line 102. The container connector 105 may, for example, be a piercing device such as a hollow mandrel that may be used to pierce a septum that closes the container and typically comprises a plurality of channels therein. Such a piercing device is commonly referred to as a “spike”. Other systems are also known for connecting the drip chamber 108 to the container, such as coupling systems that do not allow the drip chamber and container to be separated once they have been connected.

The drip chamber 108 and the container connector 105 are understood to be part of the first supply line 102. Accordingly, the container connector 105 forms the end of the first supply line 102 remote from the branch 302.

A liquid-retaining filter membrane 106 is disposed in the region of the outlet 110 of the drip chamber 108. Fluid flowing through the first supply line 102 must flow through the liquid-retaining filter membrane 106.

Preferably, the liquid-retaining filter membrane 106 is a porous and in particular hydrophilic membrane, and preferably an air-stop membrane.

The liquid-retaining filter membrane property is achieved, for example, in that the membrane has a porous structure and the liquid flowing through the membrane flows through the pores or through the channels formed by the pores, wherein the abovementioned capillary effect (“capillary stop flow”) occurs.

In further embodiments, the first supply line 102 comprises a pipe in portions or as a whole instead of a tube.

In further embodiments, the liquid-retaining filter membrane 106 may alternatively be disposed at a different location of the first supply line 102. In further embodiments, in addition to the liquid-retaining filter membrane 106 in the region of the outlet 110 of the drip chamber, a further liquid-retaining filter membrane 106 may additionally be arranged at a different location of the first supply line 102.

In alternative embodiments, the drip chamber of the first supply line 102 is not provided. The line of the first supply line 102, which is in particular a tube, is then to be connected directly to the container without the interposition of a drip chamber, for example by a spike or a suitable coupling system. In this case, the liquid-retaining filter membrane 106 is arranged at a suitable location of the first supply line 102. Also in this case, a fluid flowing through the first supply line 102 must flow through the liquid-retaining filter membrane 106.

A drip chamber provides communication between the conduit and the container, as described. Typically, devices that provide aeration of the system are integrated into the drip chamber. For this purpose, the drip chamber usually comprises a vent device with a manually operated or an automatic vent valve and an aeration channel open to the interior of the drip chamber. A vent device is not shown in the FIGURE. In the prior art, different embodiments of the vent device based on different types of valves and with or without a vent filter are known, for example, manual vent devices that comprise a manually operated flap as a vent valve and automatic vent valves that comprise a check valve as a vent valve. Alternatively, the manual or automatic vent device is not integrated into a drip chamber but is located at another suitable position of the system. The present invention is compatible with manual and automatic vent devices but is not limited to the presence of a vent device.

In particular, if the container in which the liquid to be administered is provided is collapsible, the use of a drip chamber or vent device may also be omitted because it is then not necessary to allow air to enter the system for pressure equalization.

The breakdown pressure of the liquid-retaining filter membrane of the first supply line 102 is referred to as p_{Mem} or synonymously as p_{Mem}⁽ⁿ⁾.

The second supply line 202 optionally comprises a drip chamber 208, wherein reference is made to the above description of the optional drip chamber 108 of the first supply line 102 for details of this optional drip chamber 208.

The second supply line 202 further comprises an optional container connector 205 for connecting the second supply line 202 to another container 201 (second container 201). With respect to the details of this optional container connector 205, reference is made to the above description of the container connector 105 of the first supply line 102.

In the embodiment shown in the FIGURE, the second supply line 202 optionally comprises a liquid-retaining filter membrane 206 associated with the second supply line 202. This liquid-retaining filter membrane 206 is arranged, for example, in the region of the outlet 210 of the drip chamber 208 of the second supply line 202, provided that the second supply line 202 comprises a drip chamber 208. Regardless of its arrangement, a fluid flowing through the second supply line 202 flows through the liquid-retaining filter membrane 206 associated with the second supply line 202 if the second supply line comprises a liquid-retaining filter membrane 206. In particular, the second supply line 202 comprises a liquid-retaining filter membrane 206 if there is at least one other supply line in addition to the first supply line 102 and the second supply line 202.

The second supply line 202 comprises a check valve 207.

In operation, fluid is present in the second supply line 202. For example, after priming the infusion set or transfusion set, liquid is present in the second supply line 202.

The pressure then present in the second supply line 202 on the side 208 of the check valve 207 facing away from the branch 302 is referred to as p_{A}. The side 208 of the check valve 207 facing away from the branch 302 is the side of the check valve 207 upstream of the check valve 207 as viewed in the direction of a flow of fluid through the second supply line 202 to the patient connection 100. In other words, the side 208 of the check valve 207 facing away from the branch 302 corresponds to the inlet of the check valve 207 for a flow of fluid through the second supply line 202 to the patient connection 100.

The pressure then present in the second supply line 202 on the side 209 of the check valve 207 facing towards the branch 302 is referred to as p_{Z}. The side 209 of the check valve 207 facing towards the branch 302 is the side of the check valve 207 downstream of the check valve 207 as viewed in the direction of a fluid flow through the second supply line 202 to the patient connection 100. In other words, the side 209 of the check valve 207 facing towards the branch 302 corresponds to the outlet of the check valve 207 for a fluid flow through the second supply line 202 to the patient connection 100.

The check valve 207 is closed to a passage of a fluid in the direction from the branch 302. That is, the check valve 207 prevents fluid from flowing back through the second supply line 202. Flowing back is understood to mean flowing in a direction opposite to the direction of flow of the fluid when administered to the patient.

Further, the check valve 207 is closed to fluid passage in the direction towards the branch 302 if the pressure difference Δp=p_{A}−p_{Z} is less than a threshold value p_{R}.

Further, the check valve 207 is opened for fluid passage in the direction towards the branch 302 if the pressure difference Δp=p_{A}−p_{Z} is greater than a threshold value p_{R}.

The threshold value p_{R} thus corresponds to the valve opening pressure, i.e., the pressure difference between the inlet and outlet sides of the valve required to open the valve.

For example, the threshold p_{R} is set to be 50 mbar less than the breakdown pressure p_{Mem} of the liquid-retaining filter membrane 106 of the first supply line 102. The threshold p_{R} is set, for example, when the check valve 207 is manufactured. That is, the check valve 207 is manufactured to have the predetermined threshold p_{R}. It is also possible that the valve opening pressure is variable and has been set by the manufacturer or by the user to an appropriate threshold p_{R}.

p_{R} and p_{Mem} are matched such that p_{R} is less than the breakdown pressure p_{Mem}.

The check valve 207 is configured to allow fluid to pass through the check valve and thus through the second supply line 202 only under a certain condition (Δp>p_{R}) in one flow direction and to prevent the passage of fluid otherwise. The passage of fluid is prevented if this condition is not met, and in particular in the direction opposite to said flow direction. The flow direction is the direction from the check valve to the branch 302.

The first supply line 102 may optionally also comprise a check valve 107. This check valve 107 associated with the first supply line 102 is disposed between the branch 302 and the fluid retaining membrane 106. This check valve 107 is closed to a passage of a fluid in the direction from the branch 302. In particular, this check valve 107 has the advantage that during and after priming of the infusion set or transfusion set 1, an unintentional backflow of the liquid through the first supply line 102 towards the container 101 may be prevented.

Operation of the infusion set or transfusion set 1 according to the first embodiment or of a system according to the invention comprising the infusion set or transfusion set 1 according to the first embodiment, is described below for a preferred method of use in the context of sequential infusion of two liquids.

Prior to administering the liquids to the patient, the infusion set or transfusion set 1 is subjected to priming. For this purpose, the ends 105, 205 of the supply lines 102, 202 or the container connectors 105, 205 forming these ends are connected to a first container 101 containing the first liquid to be administered and to a second container 201 containing the second liquid to be administered after the first liquid, respectively. Next, the conduit system is filled with liquid, preferably the first supply line 102 and the discharge line 303 are filled with the first liquid and the second supply line 202 is filled with the second liquid. Before or after priming, the patient access port (not shown in the FIGURE) is connected to the patient connection 100. After priming, the pump 301 begins pumping the liquid entering the first supply line 102 from the first container 101 through the branch 302, the discharge line 303, and the patient connection into the patient access port, through which it enters the patient's body.

Herein, the pressure difference Δp=p_{A}−p_{Z} is less than the predetermined threshold p_{R}. To ensure this, the second container 202 should not be located significantly higher than the first container 101, otherwise the height difference may provide a disturbing contribution to the pressure difference Δp.

The check valve 207 is closed to fluid passage in the direction towards the branch 302 because Δp is less than p_{R}. Therefore, no liquid may flow from the second container 201 through the second supply line 202 and further to the patient connection 100 via the branch 302 and the discharge line 303.

Thus, as long as liquid flows through the first supply line 102, the following inequalities apply:

Δp<p_{R}<p_{Rmax}<p_{Mem},

or, written with the appropriate indices, which are not required for the case of two supply lines:

Δp⁽²⁾<p_{R}⁽²⁾<p_{Rmax}<p_{Mem}⁽¹⁾.

When the liquid of the first container 101 is used up, the liquid level in the first supply line 102 decreases until the liquid level reaches the liquid-retaining filter membrane 106 associated with the first supply line, which is arranged, for example, at the bottom of a drip chamber 108 of the first supply line 102. As a result of the above-described function and features of the liquid-retaining filter membrane 106, the liquid may not flow further through the first supply line 102, and no gas, i.e., air, may flow through the liquid-retaining filter membrane 106 associated with the first supply line 102. A pressure drop occurs in the fluid system downstream of the liquid-retaining filter membrane 106 associated with the first supply line 102. Therefore, p_{Z} decreases. As a result, Δp=p_{A}−p_{Z} becomes larger. Once Δp exceeds the threshold p_{R}, the check valve 207 associated with the second supply line 202 opens and liquid from the second container 201 may flow through the second supply line 202, the check valve 207, the branch 302, and the discharge line 303 to the patient connection 100. Further, due to the function and properties of the liquid-retaining filter membrane 106, neither liquid nor gas (i.e. air) may not flow through the first supply line 102. Therefore, the liquid-retaining filter membrane 106 closes off the first supply line 102.

The following inequalities apply when liquid flows through the second supply line 202:

p_{R}<Δp<p_{Mem},

or, written with the appropriate indices, which are not required for the case of two supply lines

p_{R}⁽²⁾<Δp⁽²⁾<p_{Mem}⁽¹⁾.

When the liquid in the second container 201 is also used up, the sequential infusion is complete. If the second supply line 202 also has a liquid-retaining filter membrane 206, the liquid level in the second supply line 202 decreases until the liquid level reaches the liquid-retaining filter membrane 206 associated with the second supply line 202, which is arranged, for example, at the bottom of a drip chamber 208. As a result of the above-described function and features of the liquid-retaining filter membrane 206, the liquid may not continue to flow through the second supply line 202, and no gas, i.e., air, may flow through the liquid-retaining filter membrane 206 associated with the second supply line 202. A pressure drop occurs in the fluid system downstream of the liquid-retaining filter membrane 206 associated with the second supply line 202, Δp increases as a result.

The concept underlying the above described administration of a sequential infusion or transfusion of two liquids from a first container 201 and a second container 201 using the infusion set or transfusion set 1 according to the invention may be summarized as follows: The arrangement comprising the infusion set or transfusion set 1 and the containers 101, 201 comprises two fluid systems, namely a primary system and a secondary system. Both subsystems have the function of a conventional infusion set or transfusion set and each serves to administer a liquid. The primary system includes the first container 101, the first supply line 102, the junction 302, the discharge line 303, and the patient connection 100. The primary system is for administering the liquid from the first container 101. The secondary system includes the second container 201, the second supply line 202, the junction 302, the discharge line 303, and the patient connection 100. The secondary system is for administering the liquid from the second container 201. The primary system is automatically deactivated and the secondary system is automatically activated when the first container 101 has become empty.

Optionally, the second supply line 202 comprises an element 402 having a function analogous to the branch 302 if a third supply line 502 having a check valve associated with the third supply line 502 is connected to the element 402 and a liquid-retaining filter membrane 206 associated with the second supply line 202 is provided. The element 402 is disposed between the check valve 207 associated with the second supply line 202 and the liquid-retaining filter membrane 206 associated with the second supply line 202. The second embodiment of the invention, in which the third supply line 502 is connected to the element 402, has an analogous function to the third embodiment, in which a third supply line 502 is connected to the branch 302.

The second supply line 202 may optionally comprise a further check valve 217 between the element 402 and the fluid retaining membrane 206 associated with the second supply line 202. This further check valve 217 is closed to passage of a fluid in the direction towards the fluid retaining membrane 206 associated with the second supply line 202. In particular, this further check valve 217 has the advantage that during and after priming of the infusion set or transfusion set 1, an unintentional backflow of the liquid through the second supply line 202 towards the container 201 may be prevented.

Instead of connecting the third supply line 502 by the element 402, the third supply line 502 may also be branched off directly from the second supply line.

Analogously to the connection of a third supply line 502 to the second supply line 202 by the element 402, a fourth supply line may be connected to the third supply line 502 or a fourth supply line may be branched off directly from the third supply line 502. In an analogous manner, further supply lines may be connected to or directly branched from the supply line having the respective ordinal number lower by one.

The operation of the infusion set or transfusion set 1 according to the second embodiment with a third supply line 502 connected to or directly branched off from the second supply line 202 and the infusion set or transfusion set 1 according to the third embodiment with three supply lines 102, 202, 502, all of which are connected to the branch 302, and of a system according to the invention with the infusion set or transfusion set 1 according to one of these embodiments with three supply lines 102, 202, 502 is described below for a preferred method of use in the context of the sequential infusion of three liquids.

This method of use takes place in an analogous manner in the context of the sequential infusion of more than three liquids, in which case an infusion set or transfusion set 1 with a number of supply lines (nₘₐₓ) corresponding to the number of liquids is used. The case nₘₐₓ>3 is therefore not described separately.

Before administering the liquids to the patient, the infusion set or transfusion set 1 is subjected to priming. For this purpose, the ends of the three supply lines or the container connectors forming these ends are connected to a first container 101 containing the first liquid to be administered, to a second container 201 containing the second liquid to be administered after the first liquid, and to a third container (not shown in the FIGURE) containing the third liquid to be administered after the second liquid, respectively. Next, the conduit system is filled with liquid, preferably the first supply line 102 and the discharge line 303 are filled with the first liquid, the second supply line 202 is filled with the second liquid, and the third supply line 502 is filled with the third liquid. Before or after priming, the patient access port (not shown in the FIGURE) is connected to the patient connection 100. After priming, the pump 301 begins pumping the liquid entering the first supply line 102 from the first container 101 through the branch 302, the discharge line 303, and the patient connection into the patient access port, through which it enters the patient's body.

Herein, the pressure difference Δp⁽²⁾=p_{A}⁽²⁾−p_{Z}⁽²⁾ (synonymously Δp=p_{A}−p_{Z}) is less than the predetermined threshold p_{R}⁽²⁾.

The check valve 207 associated with the second supply line 202 is closed to fluid passage in the direction towards the branch 302 because Δp⁽²⁾ is less than p_{R}⁽²⁾. Therefore, no liquid may flow from the second container 201 through the second supply line 202 and further to the patient connection 100 via the branch 302 and the discharge line 303.

p_{Mem}⁽¹⁾ and p_{Mem}⁽²⁾ may be the same or different from each other. For simplicity, it is assumed that p_{Mem}⁽¹⁾ and p_{Mem}⁽²⁾ are the same (p_{Mem}⁽¹⁾=p_{Mem}⁽²⁾=p_{Mem}). If p_{Mem}⁽¹⁾ and p_{Mem}⁽²⁾ are not equal, the smaller of the two values is to be substituted for p_{Mem} in subsequent inequalities.

As long as liquid flows through the first supply line 102, the following inequalities apply:

Δp⁽²⁾<p_{R}⁽²⁾<p_{R}⁽³⁾<p_{Rmax}<p_{Mem}.

When the liquid of the first container 101 is used up, the liquid level in the first supply line 102 decreases until the liquid level reaches the liquid-retaining filter membrane 106 associated with the first supply line 102, which is arranged, for example, at the bottom of a drip chamber 108 of the first supply line 102. As a result of the above-described function and features of the liquid-retaining filter membrane 106 associated with the first supply line 102, the liquid may not flow further through the first supply line 102, and no gas, i.e., air, may flow through the liquid-retaining filter membrane 106 associated with the first supply line 102. A pressure drop occurs in the fluid system downstream of the liquid-retaining filter membrane 106 associated with the first supply line 102. Therefore, p_{Z}⁽²⁾ decreases. As a result, Δp⁽²⁾=p_{A}⁽²⁾−p_{Z}⁽²⁾ becomes larger. Once Δp⁽²⁾ exceeds the threshold p_{R}⁽²⁾, the check valve 207 associated with the second supply line 202 opens and liquid from the second container 201 may flow through the second supply line 202, the check valve 207, the branch 302, and the discharge line 303 to the patient connection 100. Further, due to the function and features of the liquid-retaining filter membrane 106, neither liquid nor gas, i.e. air, may not flow through the first supply line 102. Therefore, the liquid-retaining filter membrane 106 closes off the first supply line 102.

The following inequalities apply when liquid flows through the second supply line 202:

p_{R}⁽²⁾<Δp⁽²⁾<p_{R}⁽³⁾<p_{Rmax}<p_{Mem}.

When the liquid of the second container 201 is used up, the liquid level in the second supply line 202 decreases until the liquid level reaches the liquid-retaining filter membrane 106 associated with the second supply line 202, which is arranged, for example, at the bottom of a drip chamber 208 of the second supply line 202. As a result of the above-described function and features of the liquid-retaining filter membrane 206 associated with the second supply line 202, the liquid may not flow further through the second supply line 202, and no gas, i.e., air, may flow through the liquid-retaining filter membrane 206 associated with the second supply line 202. A pressure drop occurs in the fluid system downstream of the liquid-retaining filter membrane 206 associated with the second supply line 202. Therefore, p_{Z}⁽³⁾ decreases. As a result, Δp⁽³⁾=p_{A}⁽³⁾−p_{Z}⁽³⁾ becomes larger. Once Δp⁽³⁾ exceeds the threshold p_{R}⁽³⁾, the check valve associated with the third supply line 502 (not shown in the FIGURE) opens and liquid from the third container (not shown in the FIGURE) may flow through the third supply line 502, the check valve associated with the third supply line, the branch 302, and the discharge line 303 to the patient connection 100. Further, due to the function and features of the liquid-retaining filter membranes 106, 206 associated with the first supply line 102 and the second supply line 202, neither liquid nor gas, i.e., air, may flow through the first supply line 102 and the second supply line 202. Therefore, the liquid-retaining filter membranes 106, 206 close off the first supply line 102 and the second supply line, respectively.

The following inequalities apply when liquid flows through the third supply line 502:

p_{R}⁽²⁾<p_{R}⁽³⁾<Δp⁽³⁾<p_{Mem}.

When the liquid in the third container is also used up, the sequential infusion is completed. If the third supply line 502 also has a liquid-retaining filter membrane, the liquid level in the third supply line 502 decreases until the liquid level reaches the liquid-retaining filter membrane associated with the third supply line 502, which is arranged, for example, at the bottom of a drip chamber (not shown in the FIGURE) of the third supply line 502. As a result of the above-described function and features of the liquid-retaining filter membrane associated with the third supply line 502, the liquid may not flow further through the third supply line 502, and no gas, i.e., air, may flow through the liquid-retaining filter membrane associated with the third supply line 502. There is a pressure drop in the fluid system downstream of the liquid-retaining filter membrane associated with the third supply line 502, Δp⁽³⁾ increases as a result.

The infusion set or transfusion set 1 may optionally include other components, such as one or more flow controllers 600, 700 for shutting off the discharge line and/or supply lines and/or controlling the flow rate of the liquid. Two optional flow regulators 600, 700 are shown as exemplary roller clamps in the FIGURE.

1. An infusion set or transfusion set for administering a liquid from a container using a pump,
wherein the infusion set or transfusion set comprises a branch,
wherein a fluid connection is provided via the branch between one end of each of a first supply line, a second supply line, and a discharge line,
wherein the first supply line is configured to be connected to the container at its end remote from the branch,
wherein at least the first supply line comprises a liquid-retaining filter membrane having a breakdown pressure p_{Mem},
wherein at least the second supply line comprises a check valve,
wherein the check valve is closed to passage of a fluid in the direction from the branch,
wherein the check valve is configured to be closed for passage of fluid in the direction towards the branch if the pressure difference Δp=p_{A}−p_{Z} between a pressure p_{A} present in the second supply line on the side of the check valve facing away from the branch and a pressure p_{Z} present in the second supply line on the side of the check valve facing towards the branch is less than a threshold value p_{R},
wherein the check valve is configured to be open for fluid passage in the direction towards the branch if the pressure difference Δp=p_{A}−p_{Z} between the pressure p_{A} present in the second supply line on the side of the check valve facing away from the branch and the pressure p_{Z} present in the second supply line on the side of the check valve facing towards the branch is greater than the threshold value p_{R}, and
wherein p_{R} is less than the breakdown pressure p_{Mem} of the liquid-retaining filter membrane.

2. The infusion set or transfusion set according to claim 1,
wherein the branch is configured as a Y-port.

3. The infusion set or transfusion set according to claim 1 for sequentially administering first the liquid as a first liquid from the container as a first container and then a further liquid as a second liquid from a further container as a second container using the pump,
wherein the first supply line is configured to be connected to the first container at its end remote from the branch, and
wherein the second supply line is configured to be connected at its end remote from the branch to the second container.

4. The infusion set or transfusion set according to claim 1,
wherein the threshold value p_{R} is predetermined to be greater than or equal to a predetermined minimum threshold value p_{Rmin} and/or
wherein the threshold value p_{R} is predetermined to be less than or equal to a predetermined maximum threshold value p_{Rmax}.

5. The infusion set or transfusion set according to claim 4,
wherein the minimum threshold value p_{Rmin} is determined to be at least 25 mbar, and/or
wherein the maximum threshold value p_{Rmax} is determined to be at most 190 mbar, and/or
wherein the maximum threshold value p_{Rmax} is determined to be less than p_{Mem} by at least 10 mbar.

6. The infusion set or transfusion set according to claim 1,
wherein the first supply line comprises a drip chamber which comprises an inlet through which the liquid from the container or first container may enter in the form of drops, and which comprises an outlet through which the liquid may flow into the remaining first supply line.

7. The infusion set or transfusion set according to claim 3,
wherein the second supply line comprises a drip chamber which comprises an inlet through which the second liquid may enter from the second container in the form of drops, and which comprises an outlet through which the second liquid may flow into the remaining second supply line.

8. The infusion set or transfusion set according to claim 1,
wherein the liquid-retaining filter membrane of the first supply line is an air-stop membrane.

9. The infusion set or transfusion set according to claim 8,
wherein the liquid-retaining filter membrane of the first supply line is a hydrophilic and/or a porous membrane.

10. The infusion set or transfusion set according to claim 1,
wherein the first supply line comprises a check valve between the branch and the liquid-retaining membrane,
wherein the check valve of the first supply line is closed to passage of a fluid in the direction from the branch.

11. The infusion set or transfusion set according to claim 1,
wherein the second supply line comprises a further liquid-retaining filter membrane having a breakdown pressure p_{Mem}⁽²⁾,
wherein a third supply line branches off from the branch or a position of the first supply line or second supply line between the branch and the liquid-retaining filter membrane of the respective supply line,
wherein the third supply line comprises a further check valve,
wherein the check valve of the third supply line is closed for a passage of a fluid in the direction from the branch,
wherein the check valve of the third supply line is configured to be closed for passage of fluid in the direction towards the branch if the pressure difference Δp⁽³⁾=p_{A}⁽³⁾−p_{Z}⁽³⁾ between a pressure p_{A}⁽³⁾ present in the third supply line on the side of the check valve of the third supply line facing away from the branch and a pressure p_{Z}⁽³⁾ present in the third supply line on the side of the check valve of the third supply line facing towards the branch is less than a threshold value p_{R}⁽³⁾, and
wherein the check valve of the third supply line is configured to be open for fluid passage in the direction towards the branch if the pressure difference Δp⁽³⁾=p_{A}⁽³⁾−p_{Z}⁽³⁾ between a pressure p_{A}⁽³⁾ present in the third supply line on the side of the check valve of the third supply line facing away from the branch and a pressure p_{Z}⁽³⁾ present in the third supply line on the side of the check valve of the third supply line facing towards the branch is greater than the threshold value p_{R}⁽³⁾,
wherein the threshold value p_{R}⁽³⁾ is predetermined to be greater than the threshold value p_{R}, and
wherein p_{R}⁽³⁾ is less than the breakdown pressure p_{Mem} of the liquid-retaining filter membrane of the first supply line and less than the breakdown pressure p_{Mem}⁽²⁾ of the liquid-retaining filter membrane of the second supply line.

12. The infusion set or transfusion set according to claim 11,
wherein at least one further supply line branches off from the branch- or positions of the supply lines between the branch and the liquid-retaining filter membranes as the n-th supply line in addition to the first, second and third supply lines,
wherein n denotes a natural number running from 4 to a predetermined maximum nₘₐₓ corresponding to the total number of supply lines,
wherein the n-th supply line comprises a further check valve associated with the n-th supply line,
wherein the check valve associated with the n-th supply line is closed for a passage of a fluid in the direction from the branch,
wherein the check valve associated with the n-th supply line is configured to be closed for passage of fluid in the direction towards the branch if the pressure difference Δp⁽ⁿ⁾=p_{A}⁽ⁿ⁾−p_{Z}⁽ⁿ⁾ between a pressure p_{A}⁽ⁿ⁾ present in the n-th supply line on the side of the check valve associated with the n-th supply line facing away from the branch and a pressure p_{Z}⁽ⁿ⁾ present in the n-th supply line on the side of the check valve associated with the n-th supply line facing towards the branch is less than a threshold value p_{R}⁽ⁿ⁾, and
wherein the check valve associated with the n-th supply line is configured to be open for fluid passage in the direction towards the branch if the pressure difference Δp⁽ⁿ⁾=p_{A}⁽ⁿ⁾−p_{Z}⁽ⁿ⁾ between a pressure p_{A}⁽ⁿ⁾ present in the n-th supply line on the side of the check valve associated with the n-th supply line facing away from the branch- and a pressure p_{Z}⁽ⁿ⁾ present in the n-th supply line on the side of the check valve associated with the n-th supply line facing towards the branch is greater than the threshold value p_{R}⁽ⁿ⁾, and
wherein the threshold value p_{R}⁽ⁿ⁾ is determined to be greater than the threshold value p_{R}⁽ⁿ⁻¹⁾,
wherein p_{R}⁽ⁿ⁾ is less than the breakdown pressure p_{Mem}, p_{Mem}⁽²⁾, . . . , p_{Mem}⁽ⁿ⁻¹⁾, of the liquid-retaining filter membranes of the first through (n−1)-th supply lines.

13. A system comprising an infusion set or transfusion set according to claim 1 and a pump for conveying liquid through the supply lines and the discharge line.

14. The system according to claim 13,
wherein the system further comprises a pressure measuring device for detecting a measured value corresponding to a pump upstream pressure of a conveyed liquids.

15. The infusion set or transfusion set according to claim 6,
wherein the liquid-retaining filter membrane is arranged between the inlet and the outlet of the drip chamber.

16. The infusion set or transfusion set according to claim 15,
wherein the liquid-retaining filter membrane is arranged in an outlet region of the drip chamber.

17. The infusion set or transfusion set according to claim 7,
wherein a further liquid-retaining filter membrane is arranged between an inlet and an outlet of the drip chamber of the second supply line.

18. The infusion set or transfusion set according to claim 17,
wherein the further liquid-retaining filter membrane is arranged in an outlet region of the drip chamber of the second supply line.

19. The system according to claim 13,
wherein the pump is a peristaltic pump.

20. The system according to claim 19,
wherein the peristaltic pump is engaged with or configured to be engaged with a portion of an outer wall of the discharge line.

21. The system according to claim 14,
wherein the system is configured to send an alarm signal and/or to stop conveying liquid by the pump when the pump upstream pressure falls below a threshold value p_{Alarm}.

22. The system according to claim 21,
wherein the threshold value p_{Alarm} is selected such that no value Δp⁽ⁿ⁾ (n=2, 3, . . . , nₘₐₓ) exceeds a breakdown pressure p_{Mem}⁽ⁿ⁾ (n=1, 3, . . . , nₘₐₓ) when the pump upstream pressure falls below the threshold value p_{Alarm}.
